(19) United States
(12) Patent Application Publication
(10) Pub. No.: US 2008/0221374 A1
(43) Pub. Date: Sep. 11, 2008

(54) Method for the Production of Propene from Propane

(75) Inventors: Sven Crone, Limburgerhof (DE); Otto Machhammer, Mannheim (DE); Gotz-Peter Schindler, Mannheim (DE); Frieder Borgmeier, Mannheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/813,360

(22) PCT Filed: Jan. 4, 2006
(86) PCT No.: PCT/EP2006/000032

§ 371 Date: May 28, 2008

(30) Foreign Application Priority Data
Jan. 5, 2005 (DE) 10 2005 000 798.8
Mar. 17, 2005 (DE) 10 2005 012 291.4

(51) Int. Cl. C07C 5/02

(52) U.S. Cl. 585/252

(57) ABSTRACT

A process for preparing propene from propane, comprising the steps:
A) a feed gas stream a comprising propane is provided;
B) the fed gas stream a comprising propane, if appropriate steam and, if appropriate, an oxygenous gas stream are fed into a dehydrogenation zone and propane is subjected to a dehydrogenation to propene to obtain a product gas stream b comprising propane, propene, methane, ethane, ethene, carbon monoxide, carbon dioxide, steam, if appropriate hydrogen and, if appropriate, oxygen;
C) product gas stream b is cooled, if appropriate condensed and steam is removed by condensation to obtain a steam-depleted product gas stream c;
D) product gas stream c is contacted in a first absorption zone with a selective, inert absorbent which selectively absorbs propene to obtain an absorbent stream d1 laden substantially with propene and a gas stream d2 comprising propane, methane, ethane, ethene, carbon monoxide, carbon dioxide and hydrogen;
E) if appropriate, the absorbent stream d1 is decompressed to a lower pressure in a first desorption zone to obtain an absorbent stream e1 laden substantially with propene and a gas stream e2 comprising propene, and gas stream e2 is recycled into the first absorption zone,
F) from the absorbent stream d1 or e1 laden substantially with propene, in at least one second desorption zone, by decompression, heating and/or stripping the absorbent stream d1 or e1, a gas stream f1 comprising propene is released and the selective absorbent is recovered.

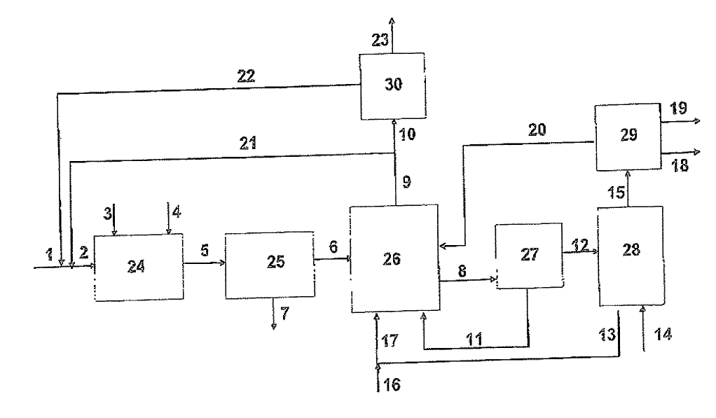

The invention relates to a process for preparing propane from propane.

Propene is obtained on the industrial scale by dehydrogenating propane.

In the process, known as the UOP-oleflex process, for dehydrogenating propane to propene, a feed gas stream comprising propane is preheated to 600-700° C. and dehydrogenated in a moving bed dehydrogenation reactor over a catalyst which comprises platinum on alumna to obtain a product gas stream comprising predominantly propane, propene and hydrogen. In addition, low-boilng hydrocarbons formed by cracking (methane, ethane, ethene) and small amounts of high boilers (C₄⁺ hydrocarbons) are present in the product gas stream. The product gas mixture is cooled and compressed in a plurality of stages. Subsequently, the C₂ and C₃ hydrocarbons and the high boilers are removed from the hydrogen and methane formed in the dehydrogenation by condensation in a “cold box”. The liquid hydrocarbon condensate is subsequently separated by distillation by removing the C₂ hydrocarbons and remaining methane in a first column and separating the C₃ hydrocarbon stream into a propene fraction having high purity and a propane fraction which also comprises the C₄⁺ hydrocarbons in a second distillation column,

A disadvantage of this process is the loss of C₃ hydrocarbons by the condensation in the cold box. Owing to the large amounts of hydrogen formed in the dehydrogenation and as a consequence of the phase equilibrium, relatively large amounts of C₃ hydrocarbons are also discharged with the hydrogen/methane offgas stream unless condensation is effected at very low temperatures. Thus, it is necessary to work at temperatures of from −20 to −60° C. in order to limit the loss of C₃ hydrocarbons which are discharged with the hydrogen/methane offgas stream.

It is an object of the present invention to provide an improved process for dehydrogenating propane to propene.

The object is achieved by a process for preparing propene from propane, comprising the steps:
A) a feed gas stream a comprising propane is provided;
B) the feed gas stream a comprising propane, if appropriate an oxygenous gas stream and, if appropriate, steam are fed into a dehydrogenation zone and propane is subjected to a dehydrogenation to propene to obtain a product gas stream b comprising propane, propene, methane, ethane, ethene, carbon monoxide, carbon dioxide, steam, if appropriate hydrogen, and, if appropriate, oxygen;
C) product gas stream b is cooled, if appropriate compressed and steam is removed by condensation to obtain a steam-depleted product gas stream c;
D) product gas stream c is contacted in a first absorption zone with a selective, inert absorbent which selectively absorbs propene to obtain an absorbent stream d1 laden substantially with propane and a gas stream d2 comprising propane, propene, methane, ethane, ethene, carbon monoxide, carbon dioxide, if appropriate hydrogen and, if appropriate, oxygen;
E) if appropriate, the absorbent stream d1 is decompressed to a lower pressure in a first desorption zone to obtain an absorbent stream e1 laden substantially with propene and a gas stream e2 comprising propene, and gas stream e2 is recycled into the first absorption zone,
F) from the absorbent stream d1 or e1 laden substantially with propene, in at least one second desorption zone, by decompression, heating and/or stripping the absorbent stream d1 or e1, a gas stream f1 comprising propene is released and the selective absorbent is recovered.

In a first process part, A, a feed gas stream a comprising propane is provided. This generally comprises at least 80% by volume of propane, preferably 90% by volume of propane. In addition, the propane-containing feed gas stream a generally also comprises butanes (n-butane, isobutane). Typical compositions of the propane-containing feed gas stream are disclosed in DE-A 102 46 119 and DR-A 102 45 585, Typically, the propane-containing feed gas stream a is obtained from liquid petroleum gas (LPG).

In one process part, B, the feed gas stream comprising propane is fed into a dehydrogenation zone and subjected to a generally catalytic dehydrogenation. In this process part, propane is dehydrogenated partially in a dehydrogenation reactor over a dehydrogenation-active catalyst to give propene. In addition, hydrogen and small amounts of methane, ethane, ethene and C₄⁺ a hydrocarbons (n-butane, isobutane, butenes, butadiene) are obtained. Also generally obtained in the product gas mixture of the catalytic propane dehydrogenation are carbon oxides (CO, CO₂), in particular CO₂, steam and, if appropriate, inert gases to a small degree. The product gas stream of the dehydrogenation comprises generally steam which has already been added to the dehydrogenation gas mixture and/or in the case of dehydrogenation in the presence of oxygen (oxidative or nonoxidative), is formed in the dehydrogenation, When the dehydrogenation is carried out in the presence of oxygen, the inert gases (nitrogen) are introduced into the dehydrogenation zone with the oxygen-containing gas stream fed in, as long as pure oxygen is not fed in. Where an oxygen-containing gas is fed in, its oxygen content is generally at least 40% by volume, preferably at least 80% by volume, more preferably at least 90% by volume. In particular technically pure oxygen having an oxygen content of >99% is fed in, in order to prevent too high an inert gas fraction in the product gas mixture. In addition, unconverted propane is present in the product gas mixture.

The propane dehydrogenation may in principle be carried out in any reactor types known from the prior art. A comparatively comprehensive description of reactor types suitable in accordance with the invention is also contained in “Catalytica® Studies Division, Oxidative Dehydrogenation and Alternative Dehydrogenation Processes” (Study Number 4192 OD, 19937 430 Ferguson Drive, Mountain View, Calif., 94043-5272, USA).

The dehydrogenation may be carried out as an oxidative or nonoxidative dehydrogenation. The dehydrogenation may be carried out isothermally or adiabatically. The dehydrogenation may be carried out catalytically in a fixed bed, moving bed or fluidized bed reactor.

The nonoxidative catalytic propane dehydrogenation is preferably carried out autothermally. To this end, oxygen is additionally admixed with the reaction gas mixture of the propane dehydrogenation in at least one reaction zone and the hydrogen and/or hydrocarbon present in the reaction gas mixture is at least partly combusted, which directly generates in the reaction gas mixture at least some of the heat required for dehydrogenation in the at least one reaction zone.

One feature of the nonoxidative method compared to an oxidative method is the at least intermediate formation of hydrogen, which is reflected in the presence of hydrogen in the product gas of the dehydrogenation. In the oxidative dehydrogenation, free hydrogen is not found in the product gas of the dehydrogenation.

A suitable reactor form is the fixed bed tubular or tube bundle reactor. In these reactors, the catalyst (dehydrogenation catalyst and if appropriate a specialized oxidation catalyst) is disposed as a fixed bed in a reaction tube or in a bundle of reaction tubes. Customary reaction tube internal diameters are from about 10 to 15 cm. A typical dehydrogenation tube bundle reactor comprises from about 300 to 1000 reaction tubes. The internal temperature in the reaction tubes typically varies in the range from 300 to 1200° C., preferably in the range from 500 to 1000° C. The working pressure is customarily from 0.5 to 8 bar, frequently from 1 to 2 bar, when a low steam dilution is used, or else from 3 to 8 bar when a high steam dilution is used (corresponding to the steam active reforming process (STAR process) or the Lined process) for the dehydrogenation of propane or butane of Phillips Petroleum Co. Typical gas hourly space velocities (GHZ) are from 500 to 2000 h⁻¹, based on hydrocarbon used. The catalyst geometry may, for example, be spheric or cylindrical (hollow or solid).

The catalytic propane dehydrogenation may also be carried out under heterogeneous catalysis in a fluidized bed, according to the Snamprogetti/Yarsintez-FBD process. Appropriately, two fluidized beds are operated in parallel, of which one is generally in the state of regeneration.

The working pressure is typically from 1 to 2 bar, the dehydrogenation temperature generally from 550 to 600° C. The heat required for the dehydrogenation can be introduced into the reaction system by preheating the dehydrogenation catalyst to the reaction temperature. The admixing of a cofeed comprising oxygen allows the preheater to be dispensed with and the required heat to be generated directly in the reactor system by combustion of hydrogen and/or hydrocarbons in the presence of oxygen, If appropriate, a cofeed comprising hydrogen may additionally be admixed.

The catalytic propane dehydrogenation may be carried out in a tray reactor. When the dehydrogenation is carried out autothermally with feeding of an oxygen-containing gas stream, it is preferably carried out in a tray reactor. This reactor comprises one or more successive catalyst beds. The number of catalyst beds may be from 1 to 20, advantageously from 1 to 6, preferably from 1 to 4 and in particular from 1 to 3. The catalyst beds are preferably flowed through radially or axially by the reaction gas. In general, such a tray reactor is operated using a fixed catalyst bed. In the simplest case, the fixed catalyst beds are disposed axially in a shaft furnace reactor or in the annular gaps of concentric cylindrical grids. A shaft furnace reactor corresponds to one tray. The performance of the dehydrogenation in a single shaft furnace reactor corresponds to one embodiment. In a further, preferred embodiment, the dehydrogenation is carried out in a tray reactor having 3 catalyst beds.

In general, the amount of the oxygenous gas added to the reaction gas mixture is selected in such a way that the amount of heat required for the dehydrogenation of the propane is generated by the combustion of the hydrogen present in the reaction gas mixture and of, if appropriate, hydrocarbons present in the reaction gas mixture and/or of carbon present in the form of coke. In general, the total amount of oxygen supplied, based on the total amount of propane, is from 0.001 to 0.8 mol/mol, preferably from 0.001 to 0.6 mol/mol, more preferably from 0.02 to 0.5 mol/mol, Oxygen may be used either in the form of pure oxygen or in the form of oxygenous gas which comprises inert gases. In order to prevent high propane and propene losses ill the workup (see below), it is essential, however, that the oxygen content of the oxygenous gas used is high and is at least 40% by volume, preferably at least 80% by volume, more preferably at least 90% by volume. A particularly preferred oxygenous gas is oxygen of technical-grade purity with an O₂ content of approx. 99% by volume.

The hydrogen combusted to generate heat is the hydrogen formed in the catalytic propane dehydrogenation and also, if appropriate, hydrogen additionally added to the reaction gas mixture as hydrogenous gas. The amount of hydrogen present should preferably be such that the molar H₂/O₂ ratio in the reaction gas mixture immediately after the oxygen is fed in is from 1 to 10 mol/mol, preferably from 2 to 5 mol/mol. In multistage reactors, this applies to every intermediate feed of oxygenous and, if appropriate, hydrogenous gas.

The hydrogen is combusted catalytically. The dehydrogenation catalyst used generally also catalyzes the combustion of the hydrocarbons and of hydrogen with oxygen, so that in principle no specialized oxidation catalyst is required apart from it. In one embodiment, operation is effected in the presence of one or more oxidation catalysts which selectively catalyze the combustion of hydrogen to oxygen in the presence of hydrocarbons. The combustion of these hydrocarbons with oxygen to give CO, CO₂ and water therefore proceeds only to a minor extent. The dehydrogenation catalyst and the oxidation catalyst are preferably present in different reaction zones.

When the reaction is carried out in more than one stage, the oxidation catalyst may be present only in one, in more than one or in all reaction zones.

Preference is given to disposing the catalyst which selectively catalyzes the oxidation of hydrogen at the points where there are higher partial oxygen pressures than at other points in the reactor, in particular near the feed point for the oxygenous gas. The oxygenous gas and/or hydrogenous gas may be fed in at one or more points in the reactor.

In one embodiment of the process according to the invention, there is intermediate feeding of oxygenous gas and of hydrogenous gas upstream of each tray of a tray reactor. In a further embodiment of the process according to the invention, oxygenous gas and hydrogenous gas are fed in upstream of each tray except the first tray. In one embodiment, a layer of a specialized oxidation catalyst is present downstream of every feed point, followed by a layer of the dehydrogenation catalyst. In a further embodiment, no specialized oxidation catalyst is presents The dehydrogenation temperature is generally from 400 to 1100° C.; the pressure in the last catalyst bed of the tray reactor is generally from 0.2 to 15 bar, preferably from 1 to 10 bar, more preferably from 1 to 5 bar. The GHSV is generally from 500 to 2000 h⁻¹, and, in high-load operation, even up to 100 000 h⁻¹, preferably from 4000 to 16 000 h⁻¹.

A preferred catalyst which selectively catalyzes the combustion of hydrogen comprises oxides and/or phosphates selected from the group consisting of the oxides and/or phosphates of germanium, tin, lead, arsenic, antimony and bismuth. A further preferred catalyst which catalyzes the combustion of hydrogen comprises a noble metal of transition group VIII and/or I of the periodic table.

The dehydrogenation catalysts used generally have a support and an active composition. The support generally consists of a heat-resistant oxide or mixed oxide. The dehydrogenation catalysts preferably comprise a metal oxide which is selected from the group consisting of zirconium dioxide, zinc oxide, aluminum oxide, silicon dioxide, titanium dioxide, magnesium oxide, lanthanum oxide, cerium oxide and mixtures thereof, as a support. The mixtures may be physical mixtures or else chemical mixed phases such as magnesium aluminum oxide or zinc aluminum oxide mixed oxides. Preferred supports are zirconium dioxide and/or silicon dioxide, and particular preference is given to mixtures of zirconium dioxide and silicon dioxide.

Suitable catalyst molding geometries are extrudates, stars, rings, saddles, spheres, foams and monoliths having characteristic dimensions of from 1 to 100 mm.

The active composition of the dehydrogenation catalysts generally comprises one or more elements of transition group VIII of the periodic table, preferably platinum and/or palladium, more preferably platinum. Furthermore, the dehydrogenation catalysts may comprise one or more elements of main group I and/or II of the periodic table, preferably potassium and/or cesium. The dehydrogenation catalysts may further comprise one or more elements of transition group III of the periodic table including the lanthanides and actinides, preferably lanthanum and/or cerium. Finally, the dehydrogenation catalysts may comprise one or more elements of main group III and/or IV of the periodic table, preferably one or more elements from the group consisting of boron, gum, silicon, germanium, tin and lead, more preferably tin.

In a preferred embodiment, the dehydrogenation catalyst comprises at least one element of transition group VIII, at least one element of main group I and/or II, at least one element of main group III and/or IV and at least one element of transition group III including the lanthanides and actinides.

For example, all dehydrogenation catalysts which are disclosed by WO 99/46039, U.S. Pat. No. 4,788,371, EP-A 705 136, WO 99/29420, U.S. Pat. No. 5,220,091, U.S. Pat. No. 5,430,220, U.S. Pat. No. 5,877,369, EP 0 117 146, DB-EA 199 37 106, DP-A 199 37 105 and DE-A 199 37 107 may be used in accordance with the invention. Particularly preferred catalysts for the above-described variants of autothermal propane dehydrogenation are the catalysts according to examples 1, 2, 3 and 4 of DE-A 199 37 107.

Preference is given to carrying out the autothermal propane dehydrogenation in the presence of steam. The added steam serves as a heat carrier and supports the gasification of organic deposits on the catalysts, which counteracts carbonization of the catalysts and increases the onstream time of the catalysts. This converts the organic deposits to carbon monoxide, carbon dioxide and, if appropriate, water. The dilution with steam shifts the equilibrium toward the products of dehydrogenation.

The dehydrogenation catalyst may be regenerated in a manner known per se. For instance, steam may be added to the reaction gas mixture or a gas comprising oxygen may be passed from time to time over the catalyst bed at elevated temperature and the deposited carbon burnt off. After the regeneration, the catalyst is reduced with a hydrogenous gas if appropriate.

Product gas stream b can be separated into two substreams, of which one substream is recycled into the autothermal dehydrogenation, according to the cycle gas mode described in DE-A 102 11 275 and DE-A 100 28 582.

The propane dehydrogenation may be carried out as an oxidative dehydrogenation. The oxidative propane dehydrogenation may be carried out as a homogeneous oxidative dehydrogenation or as a heterogeneously catalyzed oxidative dehydrogenation.

When the propane dehydrogenation in the process according to the invention is configured as a homogeneous oxydehydrogenation, this can in principle be carried out as described in the documents U.S. Pat. No. 3,798,283, CN-A 1,105,352, Applied Catalysis, 70 (2), 1991, p. 175 to 187, Catalysis Today 13, 1992, p. 673 to 678 and the prior application DE-A 1 96 22 331.

The temperature of the homogeneous oxydehydrogenation is generally from 300 to 700° C., preferably from 400 to 600° C., more preferably from 400 to 500° C. The pressure may be from 0.5 to 100 bar or from 1 to 50 bar. It will frequently be from 1 to 20 bar, in particular from 1 to 10 bar.

The residence time of the reaction gas mixture under oxydehydrogenation conditions is typically from 0.1 or 0.5 to 20 sec, preferably from 0.1 or 0.5 to 5 sec. The reactor used may, for example, be a tubular oven or a tube bundle reactor, for example a countercurrent tubular oven with flue gas as a heat carrier, or a tube bundle reactor with salt melt as a heat carrier.

The propane to oxygen ratio in the starting mixture to be used may be from 0.5:1 to 40:1. The molar ratio of propane to molecular oxygen in the starting mixture is preferably ≦6:1, more preferably ≦5:1. In general, the aforementioned ratio will be ≧1:1, for example ≧2:1. The starting mixture may comprise further, substantially inert constituents such as H₂O, CO₂, CO, N₂, noble gases and/or propene. Propene may be comprised in the C₃ fraction coming from the refinery. It is favorable for a homogeneous oxidative dehydrogenation of propane to propene when the ratio of the surface area of the reaction space to the volume of the reaction space is at a minimum, since the homogeneous oxidative propane dehydrogenation proceeds by a free-radical mechanism and the reaction space surface generally functions as a free radical scavenger. Particularly favorable surface materials are aluminas, quartz glass, borosilicates, stainless steel and aluminum.

When the first reaction stage in the process according to the invention is configured as a heterogeneously catalyzed oxydehydrogenation, this can in principle be carried out as described in the documents U.S. Pat. No. 4,788,371, CN-A 1,073,893 Catalysis Letters 23 (1994) 103-106, W. Zhang, Gaodeng Xuexiao Huaxue Xuebao, 14 (1993) 566, Z. Huang, Shiyou Huagong, 21 (1992) 592, WO 97/36849, DE-A 1 97 53 817, U.S. Pat. No. 3,862,256, U.S. Pat. No. 3,887,631, DE-A 1 95 30 454, U.S. Pat. No. 4,341,664, J. of Catalysis 167, 560-569 (1997), J. of Catalysis 167, 550-559 (1997), Topics in Catalysis 3 (1996) 265-275, U.S. Pat. No. 5,086,032, Catalysis Letters 10 (1991) 181-192, Ind. Eng. Chem. Res. 1996, 35, 14-18, U.S. Pat. No. 4,255,284, Applied Catalysis A: General, 100 (1993) 111-130, J. of Catalysis 148, 56-67 (1994), V. Cortés Corberán and S. Vic Bellón (Editors), New Developments in Selective Oxidation II, 1994, Elsevier Science B.V., p. 305-313, 3rd World Congress on Oxidation Catalysis R, K. Grasselli, S. T. Oyama, A. M. Gaffney and J. E. Lyons (Editors), 1997, Elsevier Science B.V., p. 375 ff. In particular, all of the oxydehydrogenation catalysts specified in the aforementioned documents may be used. The statement made for the abovementioned documents also applies to:
a) Otsuka, K.; Uragami Y.; Komatsu, T.; Hatano, M, in Natural Gas Conversion, Stud. Surf. Sci. Catal.; Holmen A.; Jens, K. -J.; Kolboe, S., Eds.; Elsevier Science: Amsterdam, 1991; Vol. 61, p 15;
b) Seshan, K; Swaan, H. M.; Smits, R. H. H.; van Ommen, J. G.; Ross, J. R. H. in New Developments in Selective Oxidation; Stud, Surf. Sci. Catal.; Centi, G.; Trifirò, F., Eds; Elsevier Science: Amsterdam 1990; Vol. 55, p 505;
c) Smits, R. H. H.; Seshan, K.; Ross, J. R. H. in New Developments in Selective Oxidation by Heterogeneous Catalysis; Stud. Surf. Sci. Catal; Ruiz, P.; Delmon, B., Eds.; Elsevier Science: Amsterdam, 1992 a; Vol. 72, p 221;
d) Smits, R. H. H.; Seshan, K.; Ross, J. R. H. Proceedings, Symposium on Catalytic Selective Oxidation, Washington D.C.; American Chemical Society: Washington, D.C., 1992 b; 1121;
e) Mazzocchia, C.; Aboumrad, C.; Daigne, C.; Tempesti, E.; Herrmann, J. M.; Thomas, G. Catal. Lett. 1991, 10, 181;
f) Bellusi, G.; Conti, G.; Perathonar, S.; Trifirò, F. Proceeding, Symposium on Catalytic Selective Oxidation, Washington, D.C.; American Chemical Society: Washington, D.C., 1991; p 1242;
g) Ind. Eng. Chem. Res. 1996, 35, 2137-2143 and
h) Symposium on Heterogeneous Hydrocarbon Oxidation Presented before the Division of Petroleum Chemistry, Inc, 211th National Meeting, American Chemical Society New Orleans, La., Mar. 24-29, 1996.

Particularly suitable oxydehydrogenation catalysts are the multimetal oxide compositions or catalysts A of DE-A 1 97 53 817, and the multimetal oxide compositions or catalysts A specified as preferred are very particularly favorable. In other words, useful active compositions are in particular multimetal oxides of the general formula I

M¹ₐMo_{1-b}M²_{b}Oₓ (I),

where
m¹=Co, Ni, Mg, Zn, Mn and/or Cu,
M²=W, V, Te, Nb, P, Cr, Fe, Sb, Ce, Sn and/or La,
a=from 0.5 to 1.5,
b=from 0 to 0.5 and
x=a number which is determined by the valency and frequency of the elements in I other than oxygen.

Further multimetal oxide compositions suitable as oxydehydrogenation catalysts are specified below:

Suitable, Mo—V—Te/Sb—Nb—O multimetal oxide catalysts are disclosed in EP-A 0 318 295, EP-A 0 529 853, EP-A 0 603 838, EP-A 0 608 836, EP-A 0 608 838, EP-A 0 895 09, EP-A 0 962 253, EP-A 1 192 987, DE-A 198 35 247, DE-A 100 51 419 and DE-A 101 19 933.

Suitable Mo—V—Nb—O multimetal oxide catalysts are described inter alia, in E. M. Thorsteinson, T. P. Wilson, F. G. Young, P, H. Kasei, Journal of Catalysis 52 (1978), pages 116-132, and in U.S. Pat. No. 4,250,346 and EP-A 294 845.

Suitable Ni—X—O multimetal oxide catalysts where X=Ti, Ta, Nb, Co, Hf, W, Y, Zn, Zr, Al, are described in WO 00/48971.

In principle, suitable active compositions can be prepared in a simple manner by obtaining from suitable sources of their components a very intimate, preferably finely divided dry mixture corresponding to the stoichiometry and calcining it at temperatures of from 450 to 1000° C. The calcination may be effected either under inert gas or under an oxidative atmosphere, for example air (mixture of inert gas and oxygen), and also under a reducing atmosphere (for example mixture of inert gas, oxygen and NH₃, CO and/or H₂). Useful sources for the components of the multimetal oxide active compositions include oxides and/or those compounds which can be converted to oxides by heating, at least in the presence of oxygen. In addition to the oxides, such useful starting compounds are in particular halides, nitrates, formates, oxalates, citrates, acetates, carbonates, amine complex salts, ammonium salts and/or hydroxides.

The multimetal oxide compositions may be used for the process according to the invention either in powder form or shaped to certain catalyst geometries, and this shaping may be effected before or after the final calcining. Suitable unsupported catalyst geometries are, for example, solid cylinders or hollow cylinders having an external diameter and a length of from 2 to 10 mm. In the case of the hollow cylinders, a wall thickness of from 1 to 3 mm is appropriate. The suitable hollow cylinder geometries are, for example, 7 mm×7 mm×4 mm or 5 mm×3 mm×2 mm or 5 mm×2 mm×2 mm (in each case length×external diameter×internal diameter). The unsupported catalyst can of course also have spherical geometry, in which case the sphere diameter may be from 2 to 10 mm.

The pulverulent active composition or its pulverulent precursor composition which is yet to be calcined may of course also be shaped by applying to preshaped inert catalyst supports. The layer thickness of the powder composition applied to the support bodies is appropriately selected within the range from 50 to 500 mm, preferably within the range from 150 to 250 mm. Useful support materials include customary porous or nonporous aluminum oxides, silicon dioxide, thorium dioxide, zirconium dioxide, silicon carbide or silicates such as magnesium silicate or aluminum silicate. The support bodies may have a regular or irregular shape, preference being given to regularly shaped support bodies having distinct surface roughness, for example spheres, hollow cylinders or saddles having dimensions in the range from 1 to 100 mm. It is suitable to use substantially nonporous, surface-rough, spherical supports of steatite whose diameter is from 1 to 8 mm preferably from 4 to 5 mm.

The reaction temperate of the heterogeneously catalyzed oxydehydrogenation of propane is generally from 300 to 600° C., typically from 350 to 500° C. The pressure is from 0.2 to 15 bar, preferably from 1 to 10 bar, for example from 1 to 5 bar Pressures above 1 bar, for example from 1.5 to 10 bar, have been found to be particularly advantageous. In general, the heterogeneously catalyzed oxydehydrogenation of propane is effected over a fixed catalyst bed. The latter is appropriately deposited in the tubes of a tube bundle reactor, as described, for example, in EP-A 700 893 and in EP-A 700 714 and the literature cited in these documents. The average residence time of the reaction gas mixture in the catalyst bed is normally from 0.5 to 20 sec. The propane to oxygen ratio in the starting reaction gas mixture to be used for the heterogeneously catalyzed propane oxydehydrogenation may, according to the invention, be from 0.5:1 to 40:1. It is advantageous when the molar ratio of propane to molecular oxygen in the starting gas mixture is ≦6:1, preferably ≦5:1. In general, the aforementioned ratio may be ≧1:1, for example 2:1. The starting gas mixture may comprise further, substantially inert constituents such as H₂O, CO₂, CO, N₂, noble gases and/or propene. In addition, C₁, C₂ and C₄ hydrocarbons may also be comprised to a small extent.

On leaving the dehydrogenation zone, product gas stream b is generally under a pressure of from 0.2 to 15 bar, preferably from 1 to 10 bar, more preferably from 1 to 5 bar, and has a temperate in the range from 300 to 700° C.

In the propane dehydrogenation, a gas mixture is obtained which generally has the following composition: from 10 to 80% by volume of propane, from 5 to 50% by volume of propene, from 0 to 20% by volume of methane, ethane, ethene and C₄⁺ hydrocarbons from 0 to 30% by volume of carbon oxides, from 0 to 70% by volume of steam and from 0 to 25% by volume of hydrogen, and also from 0 to 50% by volume of inert gases.

In the preferred autothermal propane dehydrogenation, a gas mixture is obtained which generally has the following composition: from 10 to 80% by volume of propane, from 5 to 50% by volume of propene, from 0 to 20% by volume of methane, ethane, ethene and C₄⁺ hydrocarbons, from 0.1 to 30% by volume of carbon oxides, from 1 to 70% by volume of steam and from 0.1 to 25% by volume of hydrogen, and also from 0 to 30% by volume of inert gases.

In process part C, water is initially removed from product gas stream b. The removal of water is carried out by condensation, by cooling and, if appropriate, compressing product gas stream b, and may be carried out in one or more cooling and, if appropriate, compression stages. In general, product gas steam b is cooled for this purpose to a temperature in the range from 20 to 80° C., preferably from 40 to 65° C. In addition, the product gas stream may be compressed, generally to a pressure in the range from 2 to 40 bar, preferably from 5 to 20 bar, more preferably from 10 to 20 bar.

In one embodiment of the process according to the invention, product gas stream b is passed through a battery of heat exchangers and thus initially cooled to a temperature in the range from 50 to 200° C. and subsequently cooled further in a quenching tower with water to a temperature of from 40 to 80° C., for example 55° C. This condenses out the majority of the steam, but also some of the C₄⁺ hydrocarbons present in product gas stream b, in particular the C₅⁺ hydrocarbons. Suitable heat exchangers are, for example, direct heat exchangers and countercurrent heat exchangers, such as gas-gas countercurrent heat exchangers, and air coolers.

A steam-depleted product gas stream c is obtained. This generally still comprises from 0 to 10% by volume of steam. For the virtually full removal of water from product gas stream c, when particular solvents are used in step D), drying by means of molecular sieve or membranes may be provided for.

In one process step, D), product gas stream c is contacted in a first absorption zone with a selected inert solvent which selectively absorbs propene to obtain an absorbent stream d1 laden with C₃ hydrocarbons, substantially with propene, and a gas stream d2 comprising propane, methane, ethane, ethene, carbon monoxide, carbon dioxide and hydrogen, Propene may also be present in small amounts in gas stream d2.

Before carrying out process step D), carbon dioxide can be removed from the product gas stream c by gas scrubbing to obtain a carbon dioxide-depleted product gas stream c. The carbon dioxide gas scrubbing may be preceded by a separate combustion stage in which carbon monoxide is oxidized selectively to carbon dioxide.

For the CO₂ removal, the scrubbing liquid used is generally sodium hydroxide solution, potassium hydroxide solution or an alkanolamine solution; preference is given to using an activated N-methyldiethanolamine solution. In general, before the gas scrubbing is carried out, the product gas stream c is compressed to a pressure in the range from 5 to 25 bar by compression in one or more stages.

A carbon dioxide-depleted product gas stream d having a CO₂ content of generally <100 ppm, preferably <10 ppm, is obtained.

The absorption may be effected by simply passing stream c through the absorbent. However, it may also be effected in columns. It is possible to work in cocurrent, countercurrent or cross current. Suitable absorption columns are, for example, tray columns with bubble-cap trays, valve trays and/or sieve trays, columns having structured packings, for example fabric packings or sheet metal packings having a specific surface area of from 100 to 1000 m²/m³, such as Mellapak® 250 Y, and columns having random packings, for example having spheres, rings or saddles of metal, plastic or ceramic as random packings. However, it is also possible to use trickle and spray towers, graphite block absorbers, surface absorbers such as thick-film and thin-film absorbers, and bubble columns, with and without internals.

The absorption column preferably has an absorption section and a rectification section. The absorbent is introduced generally at the top of the column, and stream c is generally fed in in the middle or the upper half of the column. To increase the propene enrichment in the solvent by the method of rectification, it is then possible to introduce heat into the column bottom. Alternatively, a stopping gas stream can be fed into the column bottom, for example composed of nitrogen, air, steam or propene, preferably of propene. A portion of the top product may be condensed and reintroduced at the top of the column as reflux in order to restrict solvent losses.

Suitable selective absorbents which selectively absorb propene are, for example, N-methyl-pyrrolidone (NMP), NMP/water mixtures comprising up to 20% by weight of water, m-cresol, acetic acid, methylpyrazine, dibromomethane, dimethylformamide (DMF), propylene carbonate, N-formylmorpholine, ethylene carbonate, formamide, malononitrile, gamma-butyrolactone, nitrobenzene, dimethyl sulfoxide (DMSO), sulfolane, pyrrole, lactic acid, acrylic acid, 2-chloropropionic acid, triallyl trimellitate, tris(2-ethylhexyl) trimellitate, dimethyl phthalate, dimethyl succinate, 3-chloropropionic acid, morpholine, acetonitrile, 1-butyl-3-methylimidazolinium octylsulfate, ethylmethylimidazolinium tosylate, dimethylaniline, adiponitrile and formic acid.

Preferred selectively absorbing absorbents are NMP, NMP/water mixtures having up to 20% by weight of water, acetonitrile, and mixtures of acetonitrile, organic solvents and/or water having an acetonitrile content of ≧50% by weight, and also dimethylaniline.

The absorption step D) is generally carried out at a pressure of from 2 to 40 bar, preferably of from 5 to 20 bar, more preferably of from 10 to 20 bar. In addition to propene, propane is also absorbed to a certain extent by the selective absorbent. In addition, small amounts of ethene and butenes may also be absorbed.

In an optional step E), the absorbent stream d1 is decompressed to a lower pressure in a first desorption zone to obtain an absorbent stream e1 laden substantially with propene and a gas stream e2 which comprises mainly propene and still comprises small amounts of propane, and gas stream e2 is recycled into the first absorption zone, preferably as a stripping gas into the rectification section of the absorption column.

To this end, the absorbent steam d1 is decompressed from a pressure which corresponds to the pressure of the absorption stage D) to a pressure of generally from 1 to 20 bar, preferably from 1 to 10 bar. The decompression may be carried out in several stages, generally up to 5 stages, for example 2 stages. The laden absorbent stream may additionally also be heated.

A gas stream e2 comprising propene is obtained, which comprises generally from 0 to 5% by volume of propane, from 50 to 99% by volume of propene and from 0 to 15% by volume of further gas constituents such as steam, ethylene and carbon oxides, and from 0 to 50% by volume of solvent. This is recycled into the absorption zone. Preference is given to adding the recycled gas stream e2 in the lower portion of the absorption column, for example at the height of the 1st-10th theoretical plate. As a result of the recycled propene stream, propane dissolved in the absorbent is stripped out and the degree of propene enrichment in the absorbent is thus increased.

In one step, F), from the absorbent stream d1 or e1 laden substantially with propene, in at least one (second) desorption zone, by decompression, heating and/or stripping the absorbent stream d1 or e1, a gas stream f1 comprising propene is released and the selective absorbent is recovered. If appropriate, a portion of this absorbent stream which may comprise C₄⁺ hydrocarbons is discharged, worked up and recycled, or discarded.

To desorb the gases dissolved in the absorbent, it is heated and/or decompressed to a lower pressure. Alternatively, the desorption may also be effected by stripping, typically with steam, or in a combination of decompression, heating and stripping, in one or more process steps.

The gas stream f1 which comprises propene and has been released by desorption comprises generally, based on the hydrocarbon content, at least 98% by volume of propene, preferably at least 99% by volume of propene, more preferably at least 99.5% by volume of propene. In addition, it may comprise from 0 to 2% by volume of propane and small amounts of low-boiling hydrocarbons such as methane and ethene, but generally not more than 0.5% by volume, preferably not more than 0.2% by volume. When desorption is effected by stripping with steam, gas stream f1 also comprises steam generally in amounts of up to 50% by volume based on the entire gas stream.

When propane is desorbed in process part E by stripping with steam, the steam is generally subsequently removed again from gas stream f1. This removal may be effected by condensation, by cooling and, if appropriate, compression of gas stream f1. The removal may be carried out in one or more cooling and, if appropriate, compression stages.

In general, gas stream f1 is cooled for this purpose to a temperature in the range from 0 to 80° C., preferably from 10 to 65° C. In addition, the product gas stream may be compressed, for example to a pressure in the range from 2 to 50 bar. To virtually fully remove water from gas stream f1, a drying by means of molecular sieve may be provided for. The drying may also be effected by adsorption, membrane separation, rectification or further drying processes known from the prior art.

In order to achieve a particularly high propene content of gas stream f1, preference is given to recycling a portion of the gas stream f1 which comprises propene and is obtained in step F) into the absorption zone. The proportion of the recycled gas stream is generally from 0 to 25%, preferably from 0 to 10%, of gas stream f1.

In general, at least a portion of the propane present in gas stream d2 is recycled into the dehydrogenation zone.

In one embodiment of the process according to the invention, the gas stream d2 comprising propane is recycled at least partly directly into the dehydrogenation zone, and the substream (purge gas stream) is generally removed from gas stream d2 to discharge inert gases, hydrogen and carbon oxide. The purge gas stream may be incinerated. However, a substream of gas stream d2 may be recycled directly into the dehydrogenation zone, and propane may be removed by absorption and desorption from a further substream and recycled into the dehydrogenation zone.

In a further preferred embodiment of the process according to the invention, at least a portion of the gas stream d2 which comprises propane and is obtained in step D) is contacted with a high-boiling absorbent in a further step G) and the gases dissolved in the absorbent are subsequently desorbed to obtain a recycled stream g1 consisting substantially of propane and an offgas stream g2 comprising methane, ethane, ethene, carbon monoxide, carbon dioxide and hydrogen. The recycle stream consisting substantially of propane is recycled into the first dehydrogenation zone.

To this end, in an absorption stage, gas stream d2 is contacted with an inert absorbent to absorb propane and also small amounts of the C₂ hydrocarbons in the inert absorbent and obtain an absorbent laden with propane and an offgas comprising the remaining gas constituents. Substantially, these are carbon oxides, hydrogen, inert gases and C₂ hydrocarbons and methane. In a desorption stage, propane is released again from the absorbent.

Inert absorbents used in the absorption stage are generally high-boiling nonpolar solvents in which the propane to be removed has a distinctly higher solubility than the remaining gas constituents. The absorption may be effected by simply passing stream d2 through the absorbent. However, it may also be effected in columns or in rotary absorbers. It is possible to work in cocurrent, countercurrent or crosscurrent. Suitable absorption columns are, for example, tray columns having bubble-cap trays, centrifugal trays and/or sieve trays, columns having structured packings, for example fabric packings or sheet metal packings having a specific surface area of from 100 to 1000 m²/m³ such as Mellapak® 250 Y, and columns having random packing. It is also possible to use trickle and spray towers, graphite block absorbers, surface absorbers such as thick-film and thin-film absorbers, and also rotary columns, pan scrubbers, cross-spray scrubbers, rotary scrubbers and bubble columns with and without internals.

Suitable absorbents are comparatively nonpolar organic solvents, for example aliphatic C₄-C₁₈-alkenes, naphtha or aromatic hydrocarbons such as the middle oil fractions from paraffin distillation, or ethers having bulky groups, or mixtures of these solvents, to which a polar solvent such as dimethyl 1,2-phthalate may be added. Suitable absorbents are also esters of benzoic acid and phthalic acid with straight-chain C₁-C₈-alkanets, such as n-butyl benzoate, methyl benzoate, ethyl benzoate, dimethyl phthalate, diethyl phthalate, and also heat carrier oils such as biphenyl and biphenyl ether, chlorine derivatives thereof, and triaryl alkenes. A suitable absorbent is a mixture of biphenyl and biphenyl ether, preferably in the isotropic composition, for example the commercially available Diphyl®. Frequently, this solvent mixture comprises dimethyl phthalate in an amount of from 0.1 to 25% by weight. Suitable absorbents are also butanes, pentanes, hexanes, heptanes, octanes, nonanes, decanes, undecanes, dodecanes, tridecanes, tetradecanes, pentadecanes, hexadecanes, heptadecanes and octadecanes, or fractions which are obtained from refinery streams and comprise the linear alkenes mentioned as main components.

To desorb propane, the laden absorbent is heated and/or decompressed to a lower pressure. Alternatively, the desorption may also be effected by stripping, typically with steam or an oxygenous gas, or in a combination of decompression, heating and stripping, in one or more process steps. For example, the desorption may be carried out in two stages, the second desorption stage being carried out at a lower pressure than the first desorption stage and the desorption gas of the first stage being recycled into the absorption stage. The absorbent regenerated in the desorption stage is recycled into the absorption stage.

In one process variant, the desorption step is carried out by decompressing and/or heating the laden desorbent. In a further process variant, stripping is effected additionally with steam. In a further process variant, stripping is effected additionally with an oxygenous gas. The amount of the stripping gas used may correspond to the oxygen demand of the autothermal dehydrogenation.

Alternatively, in process step G), carbon dioxide may be removed by gas scrubbing from the gas stream d2 or a substream thereof to obtain a carbon dioxide-depleted recycle stream g1. The carbon dioxide gas scrubbing may be preceded by a separate incineration stage in which carbon monoxide is oxidized selectively to carbon dioxide.

For the CO₂ removal, generally sodium hydroxide solution, potassium hydroxide solution or an alkanolamine solution is used as the scrubbing liquid; preference is given to using an activated N-methyldiethanolamine solution. In general, before the gas scrubbing is carried out, product gas stream c is compressed by one-stage or multistage compression to a pressure in the range from 5 to 25 bar. It is possible to obtain a carbon dioxide depleted recycle stream g1 having a CO₂ content of generally <100 ppm, preferably <10 ppm.

If appropriate, hydrogen may be removed from gas stream d2 by membrane separation or pressure swing absorption.

To remove the hydrogen present in the offgas stream, the offgas stream may, if appropriate after cooling, for example in an indirect heat exchanger, be passed through a membrane, generally configured as a tube, which is permeable only to molecular hydrogen. The thus removed molecular hydrogen may, if required, be used at least partly in the dehydrogenation or else be sent to another utilization, for example to generate electrical energy in fuel cells. Alternatively, the offgas stream may be incinerated.

The invention is illustrated in detail by the example which follows.

### EXAMPLE

The variant, shown in the figure, of the process according to the invention was simulated by calculation. The process parameters which follow were assumed.

A capacity of the plant of 320 kt/a of propylene at running time 8000 h is assumed.

In addition to 98% by weight of propane, fresh propane typically comprises about 2% by weight of butane. The butane content could be depleted to 0.01% by weight in a C3/C4 separating column with 40 theoretical plates at an operating pressure of 10 bar and a reflux ratio of 0.41. For the fresh propane stream 1, a propane content of 100% is assumed below.

The fresh propane stream 1 is combined with the recycled streams 21 and 22 to give the propane feed stream 2. The propane stream 2 is preheated to 400° C., enters the dehydrogenation zone 24 under a pressure of approx. 3 bar and is subjected to an autothermal dehydrogenation. Also fed into the dehydrogenation zone 24 are a stream of pure oxygen 3 and a steam stream 4. The conversion of the dehydrogenation is, based on propane, 35.3%; the selectivity of propene formation is 95.5%. In addition, 0.8% cracking products (ethane and ethene) and 3.7% carbon oxides are formed by total combustion. The water concentration in the exit gas 5 of the dehydrogenation zone is 21% by weight; the residual oxygen content in the exit gas is 0% by weight; the exit temperature of the product gas mixture is 595° C.

The exit gas is cooled to 55° C. at 2.5 bar and water is condensed out down to the saturation vapor pressure. Subsequently, the product gas mixture is compressed in two stages in a two-stage compressor 25 with intermediate cooling. In the first compressor stage, compression is effected from 2.5 bar to 6 bar and in the second compressor stage from 5.9 bar to 15.3 bar, After the first compressor stage, the gas mixture is cooled to 55° C. and, after the second compressor stage, to 30° C. When this is done, a condensate stream 7 consisting substantially of water is obtained. The compressed and cooled gas stream 6 is contacted in the absorption column 26 with a water/NMP mixture 17 as the absorbent at a pressure of 15 bar. The absorbent 17 is introduced at the top of the column. The propene-laden bottom draw stream 8 of the absorption column 26 comprises only small amounts of propane, so that a propane/propene separation in the further course of the workup can be dispensed with. The propane-containing top draw stream 9 of the absorption column 26 is partly recycled as stream 21 into the dehydrogenation zone 24. The remaining substream 10 is contacted in the absorption/desorption unit 13 with tetradecane (TDC) as the absorbent. The remaining residual gas stream 23 comprises predominantly hydrogen and carbon oxides. Desorption affords a gas stream 22 which comprises predominantly propane and is recycled into the dehydrogenation zone 24. The bottom draw stream 8 composed of propene-laden absorbent is decompressed in a first desorption stage 27 to a pressure of 6 bar. When this is done, a gas stream 11 comprising predominantly propene is released and is recycled into the absorption column 26. The propene-laden absorbent is fed as stream 12 to a desorption column 28. In the desorption column 28, decompression to a pressure of 1.2 bar, heating of the bottoms and stripping with 16 bar high-pressure steam 14 desorbs propene to obtain a stream 13 composed of regenerated absorbent and a stream 15 composed of propene and steam. The regenerated absorbent 13 is supplemented by fresh absorbent 16 and recycled into the absorption column 26. The stream 15 drawn off via the top of the column is compressed to 15 bar in several stages and at the same time cooled to 40° C. in stages. When this is done, water condenses out and is discharged from the process as wastewater stream 18, and a virtually water-free pure propene stream 19 is obtained. A steam-depleted pure propene stream 20 is recycled into the absorption column.

The composition of the streams in parts by mass is reproduced by the table which follows.

TABLE

| Stream | 1 | 2 | 3 | 4 | 5 | 6 | 7 |
|---|---|---|---|---|---|---|---|
| Amount [kg/h] | 44137 | 142747 | 15962 | 24619 | 183327 | 144742 | 38585 |
| O2 | 0.0000 | 0.0000 | 1.0000 | 0.0000 | 0.0000 | 0.0000 | 0.0000 |
| H2 | 0.0000 | 0.0082 | 0.0000 | 0.0000 | 0.0098 | 0.0124 | 0.0000 |
| CO2/CO | 0.0000 | 0.0623 | 0.0000 | 0.0000 | 0.0746 | 0.0945 | 0.0000 |
| ETHENE | 0.0000 | 0.0013 | 0.0000 | 0.0000 | 0.0016 | 0.0020 | 0.0000 |
| ETHANE | 0.0000 | 0.0028 | 0.0000 | 0.0000 | 0.0034 | 0.0043 | 0.0000 |
| PROPANE | 1.0000 | 0.8675 | 0.0000 | 0.0000 | 0.4371 | 0.5535 | 0.0003 |
| PROPENE | 0.0000 | 0.0572 | 0.0000 | 0.0000 | 0.2620 | 0.3318 | 0.0003 |
| WATER | 0.0000 | 0.0006 | 0.0000 | 1.0000 | 0.2114 | 0.0015 | 0.9990 |
| NMP | 0.0000 | 0.0000 | 0.0000 | 0.0000 | 0.0000 | 0.0000 | 0.0000 |
| TDC | 0.0000 | 0.0001 | 0.0000 | 0.0000 | 0.0001 | 0.0000 | 0.0004 |
| Temperature° C. | 100.0 | 400.0 | 100.0 | 250.0 | 594.6 | 30.0 | 49.9 |
| Pressure [bar] | 3.0 | 3.0 | 3.0 | 40.0 | 2.5 | 15.3 | 15.3 |

| Stream | 8 | 9 | 10 | 11 | 12 | 13 | 14 | 15 |
|---|---|---|---|---|---|---|---|---|
| Amount [kg/h] | 2955181 | 104657 | 36630 | 222945 | 2732236 | 2689519 | 1922 | 44638 |
| O2 | 0.0000 | 0.0000 | 0.0000 | 0.0000 | 0.0000 | 0.0000 | 0.0000 | 0.0000 |
| H2 | 0.0000 | 0.0172 | 0.0172 | 0.0000 | 0.0000 | 0.0000 | 0.0000 | 0.0000 |
| CO2/CO | 0.0000 | 0.1306 | 0.1306 | 0.0000 | 0.0000 | 0.0000 | 0.0000 | 0.0000 |
| ETHENE | 0.0000 | 0.0028 | 0.0028 | 0.0000 | 0.0000 | 0.0000 | 0.0000 | 0.0000 |
| ETHANE | 0.0000 | 0.0060 | 0.0060 | 0.0000 | 0.0000 | 0.0000 | 0.0000 | 0.0000 |
| PROPANE | 0.0004 | 0.7643 | 0.7643 | 0.0041 | 0.0001 | 0.0000 | 0.0000 | 0.0031 |
| PROPENE | 0.0849 | 0.0783 | 0.0783 | 0.9348 | 0.0155 | 0.0000 | 0.0000 | 0.9502 |
| WATER | 0.0390 | 0.0008 | 0.0008 | 0.0333 | 0.0394 | 0.0400 | 1.0000 | 0.0467 |
| NMP | 0.8756 | 0.0000 | 0.0000 | 0.0279 | 0.9447 | 0.9597 | 0.0000 | 0.0000 |
| TDC | 0.0002 | 0.0000 | 0.0000 | 0.0000 | 0.0003 | 0.0003 | 0.0000 | 0.0000 |
| Temperature° C. | 72.8 | 23.7 | 23.7 | 124.1 | 124.1 | 149.3 | 200.0 | 50.0 |
| Pressure [bar] | 15.3 | 15.3 | 15.3 | 6.0 | 6.0 | 1.2 | 16.0 | 1.2 |

| Stream | 16 | 17 | 18 | 19 | 20 | 21 | 22 | 23 |
|---|---|---|---|---|---|---|---|---|
| Amount [kg/h] | 1 | 2689521 | 1968 | 40040 | 2630 | 68027 | 30583 | 6063 |
| O2 | 0.0000 | 0.0000 | 0.0000 | 0.0000 | 0.0000 | 0.0000 | 0.0000 | 0.0000 |
| H2 | 0.0000 | 0.0000 | 0.0000 | 0.0000 | 0.0000 | 0.0172 | 0.0000 | 0.1039 |
| CO2/CO | 0.0000 | 0.0000 | 0.0000 | 0.0000 | 0.0000 | 0.1306 | 0.0000 | 0.7892 |
| ETHENE | 0.0000 | 0.0000 | 0.0000 | 0.0000 | 0.0000 | 0.0028 | 0.0000 | 0.0168 |
| ETHANE | 0.0000 | 0.0000 | 0.0000 | 0.0000 | 0.0000 | 0.0060 | 0.0000 | 0.0359 |
| PROPANE | 0.0000 | 0.0000 | 0.0000 | 0.0032 | 0.0032 | 0.7643 | 0.9059 | 0.0479 |
| PROPENE | 0.0000 | 0.0000 | 0.0015 | 0.9946 | 0.9831 | 0.0783 | 0.0926 | 0.0060 |
| WATER | 0.0000 | 0.0400 | 0.9984 | 0.0021 | 0.0137 | 0.0008 | 0.0009 | 0.0000 |
| NMP | 1.0000 | 0.9597 | 0.0000 | 0.0000 | 0.0000 | 0.0000 | 0.0000 | 0.0000 |
| TDC | 0.0000 | 0.0003 | 0.0001 | 0.0000 | 0.0000 | 0.0000 | 0.0005 | 0.0002 |
| Temperature° C. | 50.0 | 45.0 | 55.0 | 40.0 | 102.3 | 150.0 | 60.7 | 50.1 |
| Pressure [bar] | 15.3 | 15.3 | 15.3 | 15.3 | 15.3 | 15.3 | 3.2 | 15.3 |

1-17. (canceled)

18. A process for preparing propene from propane, comprising the steps:
A) providing a feed gas stream (a) comprising propane;
B) feeding said feed gas stream (a) comprising propane, and optionally steam and/or an oxygenous gas stream, into a dehydrogenation zone and dehydrogenating propane to obtain a product gas stream (b) comprising propane, propene, methane, ethane, ethene, carbon monoxide, carbon dioxide, steam, and optionally hydrogen and/or oxygen;
C) cooling, optionally compressing, and removing the steam by condensation from said product gas stream (b) to obtain a steam-depleted product gas stream (c);
D) contacting said product gas stream (c) in a first absorption zone with a selective, inert absorbent which selectively absorbs propene to obtain an absorbent stream (d1) laden substantially with propene and a gas stream (d2) comprising propane, propene, methane, ethane, ethene, carbon monoxide, carbon dioxide, and optionally hydrogen and/or oxygen;
E) optionally decompressing absorbent stream (d1) to a lower pressure in a first desorption zone to obtain an absorbent stream (e1) laden substantially with propene and a gas stream (e2) comprising propene and recycling said gas stream (e2) into said first absorption zone;
F) decompressing, heating and/or stripping said absorbent streams (d1) or (e1) in at least one second desorption zone to release a gas stream (f1) comprising propene and recovering said selective absorbent.

19. The process according to claim 18, wherein said dehydrogenation is carried out as an oxidative or nonoxidative dehydrogenation.

20. The process according to claim 18, wherein said dehydrogenation is carried out adiabatically or isothermally.

21. The process according to claim 18, wherein said dehydrogenation is carried out in a fixed bed reactor, moving bed reactor or fluidized bed reactor.

22. The process according to claim 18, wherein said feed gas stream (a) comprising an oxygenous gas stream, said oxygenous gas stream comprising at least 90% by volume of oxygen.

23. The process according to claim 22, wherein said dehydrogenation is carried out as an autothermal dehydrogenation.

24. The process according to claim 18, wherein a portion of said gas stream (f1) is recycled into said first absorption zone

25. The process according to claim 18, wherein said selective absorbent is selected from the group consisting of NMP, NMP/water mixtures comprising up to 20% by weight of water, m-cresol, acetic acid, methylpyrazine, dibromomethane, DMF, propylene carbonate, N-formylmorpholine, ethylene carbonate, formamide, malononitrile, gamma-butyrolactone, nitrobenzene, DMSO, sulfolane, pyrrole, lactic acid, acrylic acid, 2-chloropropionic acid, triallyl trimellitate, tris(2-ethylhexyl) trimellitate, dimethyl phthalate, dimethyl succinate, 3-chloropropionic acid, morpholine, acetonitrile, 1-butyl-3-methylimidazolinium octylsulfate, ethylmethylimidazolinium tosylate, adiponitrile, dimethylaniline, and formic acid.

26. The process according to claim 18, wherein said first absorption zone is configured as an absorption column comprising an absorption section and a rectification section, and heat and/or a stripping gas is fed into the bottom of said absorption column.

27. The process according to claim 26, wherein step E) is performed and said absorbent stream (e1) is fed as a stripping gas into the bottom of said absorption column.

28. The process according to claim 18, wherein stripping is effected in step F) with steam.

29. The process according to claim 28, wherein said steam is condensed out of and removed as water from said gas stream (f1) by one- or multistage cooling and compression or said steam is removed by adsorption, rectification and/or membrane separation.

30. The process according to claim 18, wherein said gas stream (d2) is at least partly recycled into said dehydrogenation zone.

31. The process according to claim 18, farther comprising an additional step G) of contacting at least a portion of said gas stream (d2) with a high-boiling absorbent and subsequently desorbing the gases dissolved in said high-boiling absorbent to obtain a recycle stream (g1) consisting substantially of propane and an offgas stream (g2) comprising methane, ethane, ethene, carbon monoxide, carbon dioxide, and hydrogen, and recycling said recycle stream (g1) into said dehydrogenation zone.

32. The process according to claim 31, wherein said high-boiling absorbent is selected from the group consisting of C₄ to C₁₈ alkanes, naphtha, and the middle oil fraction from paraffin distillation.

33. The process according to claim 31, wherein said desorbing is acheived by stripping with stream.

34. The process according to claim 18, further comprising an additional step G) of removing carbon dioxide from at least a substream of said gas stream (d2) by gas scrubbing to obtain a low-carbon dioxide recycle stream (g1) and recycling said low-carbon dioxide recycle stream (g1) into said dehydrogenation zone.
